(19) United States
(12) Patent Application Publication
(10) Pub. No.: US 2005/0034180 A1
(43) Pub. Date: Feb. 10, 2005

(54) Animal integration vector and methods for its use

(76) Inventor: Patrick Fogarty, Santa Cruz, CA (US)

Correspondence Address: BOZICEVIC, FIELD & FRANCIS LLP, 1900 UNIVERSITY AVE, SUITE 200, EAST PALO ALTO, CA, 94303, US

(21) Appl. No.: 10/803,550

(22) Filed: Mar. 17, 2004

Related U.S. Application Data
(63) Continuation of application No. PCT/US02/29999, filed on Sep. 19, 2002.
Application No. PCT/US02/29999 is a continuation-in-part of application No. 09/962,853, filed on Sep. 24, 2001, now abandoned.

(51) Int. Cl. A01K 67/027; C12N 15/85

(52) U.S. Cl. 800/14; 435/455

(57) ABSTRACT

An animal integration vector and methods for its use in the insertion of an exogenous nucleic acid into the genome of a whole animal are provided. The vectors of the subject invention include a pair of transposase recognized insertion sequences, e.g., P element transposase recognized insertion sequences (such as P element derived 31 base pair inverted repeats), flanking at least one transcriptionally active gene that is located sufficienty proximal to one of the transposase recognized insertion sequences, e.g., P-feet, to provide for the desired genome integration. In practicing the subject methods, a vector as described above carrying an exogenous or endogenous nucleic acid is introduced into a target animal under conditions sufficient for entry into the animal and its cells resulting in transposition of the exogenous nucleic acid from the vector into the animal genome, i.e., in the genome of at least a portion of the cells making up the animal. The subject methods find use in a variety of transformation applications, including research, polypeptide synthesis and therapeutic applications.

### CROSS-REFERENCE TO RELATED APPLICATIONS

This application is a continuation of application serial no. PCT/US02/29999 filed on Sep. 19, 2002; which application is a continuation-in-part of U.S. application Ser. No. 09/962,853 filed on Sep. 24, 2001, the disclosure of which is herein incorporated by reference.

### INTRODUCTION

### FIELD OF THE INVENTION

The field of this invention is nucleic acid vectors.

### BACKGROUND OF THE INVENTION

Delivery of nucleic acids into a cell in culture or a whole animal is a process known as transformation. Transformation plays a major role in a variety of biotechnology and related applications, including research, synthetic and therapeutic applications. Research applications where transformation in cultured cells plays a critical role include the production of recombinant bacteria and yeast for cloning applications. Likewise, the production of transgenic animals has provided invaluable knowledge into gene function, especially C. elegans and D. melanogaster. Synthetic applications in which transformation plays a critical role include the production of peptides and proteins, where both animal and cellular systems are employed. Therapeutic applications in which transformation plays a key role include gene therapy applications, where both cellular and animal systems are employed. Because of the prevalent role transformation plays in the above and other applications, a variety of different transformation protocols have been developed.

In many transformation applications, it is desirable to introduce the exogenous DNA in a manner such that it is incorporated into a target cell's genome. One means of providing for genome integration is to employ a vector that is capable of homologous recombination. This technique is limited due to low efficiency and is applicable for cellular use only where the desired transformation event can be selected from a pool of 1000's of possibilities.

Another approach uses viral based transformation to integrate nucleic acids into the genome. Viral vectors are limited in their efficiency by the size of the nucleic acid to be delivered. Also, these viruses are limited to the species and cell type with which they can deliver nucleic acids. These limitations are for both cells and whole animals, but whole animals have further limitations. Viruses are efficiently eliminated from an animal before they are able to deliver nucleic acids. Further, if gene expression is desired from a delivered nucleic acid, this too can be problematic when using a virus to deliver into a whole animal. Many times, even if the virus successfully avoids the animal's immune response and delivers the nucleic acid into the genome, cellular mechanisms shut off the gene expression due to intracellular viral detection. Viral based vectors finding use include retroviral vectors, e.g., Maloney murine leukemia viral based vectors. Other viral based vectors that find use include adenovirus derived vectors, HSV derived vectors, sindbis derived vectors, etc. While viral vectors provide for a number of advantages, their use is not optimal in many situations.

More effective transformation vectors are continually being produced, but these are primarily for cellular transformation, primarily because of the dramatic difference in complexity when operating in a cell versus a whole animal. Accordingly, transformation vectors that integrate nucleic acids into the genome for whole animal systems are rare.

Therefore, there is continued interest in the development of new transformation vectors. Of particular interest is the development of non-viral vectors that provide for stable integration of exogenous DNA into a cellular genome in an intact animal.

Relevant Literature

U.S. Patents of interest include: U.S. Pat. Nos. 6,291,243; 5,719,055 and 4,670,388. Other references of interest include: Beall et al, Drosophila P element transposase is a novel site-specific endonuclease, Genes and Development, vol. 11, 2137-2151 (1997); Ivics et al. (1996). “Identification of functional domains and evolution of Tc1-like transportable elements” Proc. Natl. Acad. Sci., vol. 93: 5008-5013; Ivics et al. (1997). “Molecular Reconstruction of Sleeping Beauty, a Tc1-like Transposon from Fish, and its Transposition in Human Cells” Cell, vol. 91: 501-510; Luo et al. (1998). “Chromosomal transposition of a Tc1/mariner-like element in mouse embryonic stem cells” Proc. Natl. Acad. Sci., vol. 95: 10769-10773; Rio et al. (1986). “Identification and Immunochemical Analysis of Biologically Active Drosophila P Element Transposase” Cell, Vol. 44: 21-32; Rio et al. (1988). “Evidence for Drosophila P Element Transposase Activity in Mammalian Cells and Yeast” J. Mol. Biol., vol. 200: 411-415; Schouten et al. (1998). “Transposon Tc1 of the nematode Caenorhabditis elegans jumps in human cells” Nucleic Acid Research, vol. 26(12): 3013-3017; and Rio, D. C. “Regulation of Drosophilia P element transposition”, Trends in Genetics, September 1991, vol. 7, No. 9, pp. 282-287.

### SUMMARY OF THE INVENTION

An animal integration vector and methods for its use in the insertion of an exogenous nucleic acid into the genome of a whole animal are provided. The vectors of the subject invention include a pair of transposase recognized insertion sequences, e.g., P element transposase recognized insertion sequences (such as P element derived 31 base pair inverted repeats), flanking at least one transcriptionally active gene that is located sufficienty proximal to one of the transposase recognized insertion sequences, e.g., P-feet, to provide for the desired genome integration. In practicing the subject methods, a vector as described above carrying an exogenous or endogenous nucleic acid is introduced into a target animal under conditions sufficient for entry into the animal and its cells resulting in transposition of the exogenous nucleic acid from the vector into the animal genome, i.e., in the genome of at least a portion of the cells making up the animal. The subject methods find use in a variety of transformation applications, including research, polypeptide synthesis and therapeutic applications.

### BRIEF DESCRIPTION OF THE DRAWINGS

FIGS. 1A to 1G provide diagrams of the vectors recited in the Experimental Section, below.

### DESCRIPTION OF THE SPECIFIC EMBODIMENTS

Animal integration vectors and methods for their use in the insertion of an exogenous nucleic acid into the genome of a whole animal, i.e., into the genomic DNA of at least a portion of the cells that make up the animal, are provided. The subject vectors include two transposase recognized insertion sequences, e.g. P element transposase recognized insertion sequences (such as P element derived 31 base pair inverted repeats), flanking at least one transcriptionally active gene that is located in sufficient proximity to at least one of the transposase recognized insertion sequences to provide for the animal genome integration. In the subject methods, a vector according to the invention that may include an exogenous nucleic acid is administered to a whole animal under conditions sufficient for integration of the vector into the animal genome to occur. The subject methods find use in a variety of different applications, including gene therapy applications. In further describing the subject invention, the subject vectors will be described first, followed by a discussion of the methods of using the subject vectors for transformation of a target cell.

Before the subject invention is described further, it is to be understood that the invention is not limited to the particular embodiments of the invention described below, as variations of the particular embodiments may be made and still fall within the scope of the appended claims. It is also to be understood that the terminology employed is for the purpose of describing particular embodiments, and is not intended to be limiting. Instead, the scope of the present invention will be established by the appended claims. In addition, many modifications may be made to adapt a particular situation, material, composition of matter, process, process step or steps, to the objective, spirit and scope of the present invention. All such modifications are intended to be within the scope of the claims made herein.

In this specification and the appended claims, the singular forms “a,” “an” and “the” include plural reference unless the context clearly dictates otherwise. Conversely, it is contemplated that the claims may be so-drafted to exclude any optional element. This statement is intended to serve as antecedent basis for use of such exclusive terminology as “solely,” “only” and the like in connection with the recitation of claim elements or by use of a “negative” limitation Where a range of values is provided, it is understood that each intervening value, to the tenth of the unit of the lower limit unless the context clearly dictates otherwise, between the upper and lower limit of that range, and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention. Also, it is contemplated that any optional feature of the inventive variations described herein may be set forth and claimed independently, or in combination with any one or more of the features described herein.

Unless defined otherwise, all technical and scientific terms used herein have the same meaning as commonly understood to one of ordinary skill in the art to which this invention belongs. Although any methods, devices and materials similar or equivalent to those described herein can be used in the practice or testing of the invention, the preferred methods, devices and materials are now described.

All existing subject matter mentioned herein (e.g., publications, patents, patent applications and hardware) is incorporated by reference herein in its entirety. The referenced items are provided solely for their disclosure prior to the filing date of the present application. Nothing herein is to be construed as an admission that the present invention is not entitled to antedate such material by virtue of prior invention.

Vectors

In the subject vectors, a pair of transposase recognized insertion sequences (e.g., P feet (as described in greater detail below)) flank at least one transcriptionally active gene that is in approximation to, i.e., is sufficientyly close to or sufficiently proximal to, at least one of the transposase recognized insertion sequences so as to provide for the desired genomic integration. By at least one is meant one or more, usually no more than five, and more usually no more than four, where the number of transcriptionally active genes in the vector is often one, two, or three, where only one of the transcriptionally active genes need be sufficiently proximal to the transposase recognized insertion sequence. By in approximation to, i.e., sufficiently close or proximal to, is meant that the transcriptionally active gene is located at a distance from one of the transposase recognized insertion sequences that typically is less than about 7000 bp and often less than about 6,000; 5,000; 4,000; 3,000; or 2,000 bp, where in many embodiments the distance separating the transcriptionally active gene from the transposase recognized insertion sequences does not exceed about 1,000 bp. In certain embodiments, the exogenous nucleic acid that is inserted into the genome of a whole animal in the subject methods, described in greater detail infra, is one of the transcriptionally active genes of the vectors.

As indicated above, the vectors of the subject invention include a pair of transpoase recognized insertion sequences flanking at least one transcriptionally active gene or coding sequence. The transposase recognized insertion sequences may be recognized by a number of different transposases. Eukaryotic transposases have the ability to recombine DNA at specific recombination sequences, thereby excising the intervening DNA. See, Haren L, et al., Annu Rev Microbiol 53:245-81 (1999); Hallet B, et al., FEMS Microbiol Rev. 21(2):157-78 (1997). Transposases of interest include, e.g., the maize AC transposase (Haring, et al., Plant Mol Biol 16(3):449-61 (1991)), Drosophila P elements (Lankenau, Chromosoma 103(10):659-68 (1995)), insect Tcl/mariner transposons (Plasterk, Curr. Top. Microbiol. Immunol. 204:125-43 (1996); Plasterk, et al., Trends Genet 15(8):326-32 (1999)), including the fish sleeping beauty transposon (Ivics, Cell 91(4):501-10 (1997)) and yeast Ty elements (Kim, Genome Res 8(5):464-78 (1998)). Representative specific transposases of interest include, but are not limited, to: transpoases of the TC1 mariner family, e.g., Sleeping Beauty, etc.; the P-element transposase; Piggyback, TN9; and the like.

In certain embodiments of interest, the transpoase recognized insertion sequences are recognized by the P element transposase. In these embodiments, the vectors of the subject invention are P element containing vectors, i.e., P element containing transposon vectors, by which is meant that the vectors include at least the P element transposase recognized insertion sequences of the Drosophila P element. As such, the subject vectors include a pair of the 31 base pair inverted repeat domain of the P element, or the functional equivalent thereof, i.e., a domain recognized by the P element encoded transposase. The 31 base pair inverted repeat (i.e. P foot) is disclosed in Beall et al., “Drosophila P-element transposase is a novel site-specific endonuclease,” Genes Dev (Aug. 15, 1997)11(16):2137-51.

By transcriptionally active gene is meant a sequence that is at least capable of binding the transcriptional factors that initiate the transcription of DNA sequences into RNA sequences under intracellular conditions, e.g., any requisite expression regulatory elements that are required for expression in the intracellular environment. As such, the transcriptionally active genes of the subject vectors typically include a stretch of nucleotides or domain, i.e., expression module, that includes a coding sequence of nucleotides in operational combination, i.e., operably linked, with requisite trascriptional mediation or regulatory element(s). Requisite transcriptional mediation elements that may be present in the expression module include promoters, enhancers, termination and polyadenylation signal elements, splicing signal elements, and the like. In many embodiments, the transcriptionally active gene or expression module is also correctly referred to as an expression cassette.

Preferably, the expression module includes transcription regulatory elements that provide for expression of the gene in a broad host range. A variety of such combinations are known, where specific transcription regulatory elements include: SV40 elements, as described in Dijkema et al., EMBO J. (1985) 4:761; transcription regulatory elements derived from the LTR of the Rous sarcoma virus, as described in Gorman et al., Proc. Nat'l Acad. Sci USA (1982) 79:6777; transcription regulatory elements derived from the LTR of human cytomegalovirus (CMV), as described in Boshart et al., Cell (1985) 41:521; hsp70 promoters, (Levy-Holtzman, R. and I. Schechter (Biochim. Biophys. Acta (1995) 1263: 96-98) Presnail, J. K. and M. A. Hoy, (Exp. Appl. Acarol. (1994) 18: 301-308)) and the like.

In addition, the subject vectors typically include at least one restriction endonuclease recognized site. e.g., restriction site, located between the transposase recognized insertion sequences which serves as a site for insertion of an exogenous nucleic acid. A variety of restriction sites are known in the art and may be included into the vector, where such sites include those recognized by the following restriction enzymes: HindIII, PstI, SalI, AccI, HincII, XbaI, BamHI, SmaI, XmaI, KpnI, SacI, EcoRI, and the like. In many embodiments, the vector includes a polylinker, i.e., a closely arranged series or array of sites recognized by a plurality of different restriction enzymes, such as those listed above.

The inter transposase recognized insertion sequencedomain of the vectors (i.e., the space between the transposase recognized insertion sequences or that domain or region of the vector located or positioned between the transposase recognized insertion sequence (where this domain includes at least one transcriptionally active gene and the exogenous nucleic acid, when present), may vary greatly in size. Typically, the size of this inter transposase recognized insertion sequencedomain (e.g., P feet flanked domain) is at least about 50 bp in length, usually at least about 1,000 bp in length and more usually at least about 2,000 bp in length, where the length of this domain may be as long as 150,000 bp or longer, but sometimes does not exceed about 50,000 bp in length and in certain embodiments does not exceed about 20,000 bp in length.

In certain embodiments, the subject vectors further include a transposase encoding domain, i.e., a region of nucleotides having a sequence that encodes a protein having transposase activity, particularly a transposase activity that recognizes the transposase recognized insertion sequence being used, e.g., the P feet of the P element. Transposases of interest include, but are not limited to, the specific representative transposases listed above.

Incertain embodiments the protein is a protein having the P element transposase activity, i.e., P element transposase or a functional equivalent or mimetic thereof. The amino acid sequence of the P element transposase is disclosed in Rio et al., Cell (Jan. 17, 1986) 44: 21-32. A specific transposase encoding nucleic acid that may be present on the subject vectors is that found in pTURBO, where the sequence of this plasmid is disclosed in W. R. Engels, Bioess. 14: 681-686 (1992); and at the URL created by placing http://in front of “firefly.bio.indiana: and .edu at then end of this term. When present on the subject vectors, this P element transposase encoding region or domain is located outside the region flanked by the P feet. In other words, the transposase encoding region is located external to the region flanked by the P feet, i.e., outside the inter P-feet domain described supra. Put another way, the tranposase encoding region is positioned to the left of the left terminal P foot or the right of the right terminal P foot.

The subject vectors can be used to stably insert a wide variety of endogenous and/or exogenous nucleic acids into the genome of a whole animal (exogenous means a nucleic acid having a sequence that is not present in the target cell while endogenous means a nucleic acid that pre-exists in the target cell, prior to insertion). By genome of a whole animal is meant that the nucleic acid is inserted or integrated into the genomic DNA of at least a portion of the cells that make up the whole animal, e.g., at least about 5 number %, often at least about 10 number % and sometimes at least about 25 number % or more, including at least about 30, 40, 50, 60, 70, 80, 90, 95, and 99 number % or more of the cells in the whole animal or targeted organ(s) and/or tissue(s). In many embodiments, the individual cells of the animal into which the nucleic acid is integrated are distributed over a wide range of tissue types, e.g., at least about 5 different and often at least about 10 different tissue types, but may be targeted to one specific tissue or organ type. The nature of the nucleic acid will vary depending the particular protocol being performed. For example, in research applications, the exogenous or endogenous nucleic acid may be a novel gene whose protein product is not well characterized. In such applications, the vector is employed to stably introduce the gene into the genome of the target whole animal and observe changes in the phenotype in order to characterize the gene. Alternatively, in protein synthesis application, the exogenous or endogenous nucleic acid encodes a protein of interest which is to be produced by the animal. In yet other embodiments where the vector is employed as a gene therapy vector, the exogenous or endogenous nucleic acid is a gene having therapeutic activity, i.e. a gene that encodes a product of therapeutic utility. The nucleic acid may vary greatly in size. Generally, the size of the nucleic acid that is carried by the vector is at least about 50 bp, usually at least about 1000 bp and more usually at least about 2000 bp, where the length may be as long as 150,000 bp or longer. In many embodiments, the exogenous nucleic acid is long, by which is meant that it ranges in length from about 30 to 50 kb.

The subject vectors may further comprise one or more elements required for amplification of the vector in a prokaryotic host, e.g. E. coli. Elements that may be included on the vector for use in amplification of the vector in a prokaryotic host include: an origin of replication, a selectable marker, and the like.

Methods of Preparing the Subject Vectors

The vectors of the subject invention may be produced by standard methods of restriction enzyme cleavage, ligation and molecular cloning. One protocol for constructing the subject vectors includes the following steps. First, purified nucleic acid fragments containing desired component nucleotide sequences as well as extraneous sequences are cleaved with restriction endonucleases from initial sources, e.g., the Drosophila P element. Fragments containing the desired nucleotide sequences are then separated from unwanted fragments of different size using conventional separation methods, e.g., by agarose gel electrophoresis. The desired fragments are excised from the gel and ligated together in the appropriate configuration so that a circular nucleic acid or plasmid containing the desired sequences, e.g. sequences corresponding to the various elements of the subject vectors, as described above is produced. Where desired, the circular molecules so constructed are then amplified in a prokaryotic host, e.g. E. coli. The procedures of cleavage, plasmid construction, cell transformation and plasmid production involved in these steps are well known to one skilled in the art and the enzymes required for restriction and ligation are available commercially. (See, for example, R. Wu, Ed., Methods in Enzymology, Vol. 68, Academic Press, N.Y. (1979); T. Maniatis, E. F. Fritsch and J. Sambrook, Molecular Cloning: A Laboratory Manual, Cold Spring Harbor Laboratory Press, Cold Spring Harbor, N.Y. (1982); Catalog 1982-83, New England Biolabs, Inc.; Catalog 1982-83, Bethesda Research Laboratories, Inc. An example of how to construct a vector of the present invention is provided in the Experimental section, infra, and depicted in FIG. 2.

Methods of Using the Subject Vectors

The subject vectors find use in a variety of applications in which it is desired to introduce and stably integrate an exogenous or endogenous nucleic acid into the genome of a whole animal. As mentioned above, exogenous nucleic acid means a stretch of nucleotides that is not initially present in the target cell, while endogenous nucleic acid means a nucleic acid that pre-exists in the genome of the animal. In many embodiments, the sequence of nucleotides present in the exogenous nucleic acid will be one that is not found in the genome of the animal. The subject methods can be used with a variety of animals. Whole animals with which the subject vectors may be employed are generally vertebrates, where in many embodiments the animals are mammals. Of particular interest in many embodiments is the use of the subject vectors to target vertebrate animals, particularly avian and marine animals, e.g., chickens, zebrafish, and the like; mammalian animals, including murine, porcine, ovine, equine, rat, dog, cat, monkey, humans; and the like.

In the methods of the subject invention, a transposase recognized insertion sequencevector as described above is introduced into or administered to (e.g., injected into, fed to, etc.) a whole animal or directly to specific tissue(s) and/or organ(s) under conditions sufficient for excision of the transposase recognized insertion sequenceflanked nucleic acid from the vector and subsequent integration of the excised nucleic acid into the whole animal genome, as defined above, i.e., into the genomic DNA of a least a portion of the cells making up the whole animal. As the transposase recognized insertion sequencevector is introduced into the animal “under conditions sufficient for excision and integration to occur,” the subject method further includes a step of ensuring that the requisite transposase activity is present along with the introduced vector. Depending on the structure of the vector itself, i.e., whether or not the vector includes a region encoding a product having the appropriate transposase activity, the method may further include introducing a second vector into the animal which encodes the requisite transposase activity.

The vector (and second vector where necessary) may be introduced into the animal using any convenient protocol, where the protocol may provide for in vivo introduction of the vector. In vivo protocols that find use in delivery of the subject vectors include delivery via lipid based, e.g. liposome vehicles, where the lipid based vehicle may be targeted to a specific cell type for cell or tissue specific delivery of the vector. Patents disclosing such methods include: U.S. Pat. Nos. 5,877,302; 5,840,710; 5,830,430; and 5,827,703, the disclosures of which are herein incorporated by reference. Other in vivo delivery systems may also be employed, including: the use of polylysine based peptides as carriers, which may or may not be modified with targeting moieties, microinjection, electroporation, and the like. (Brooks, A. I., et al. 1998, J. neurosci. Methods V. 80 p: 137-47; Muramatsu, T., Nakamura, A., and H. M. Park 1998, Int. J. Mol. Med. V. 1 p: 55-62). Because of the multitude of different types of vectors and delivery vehicles that may be employed, administration may be by a number of different routes, where representative routes of administration include: oral, topical, intraarterial, intravenous, intraperitoneal, intramuscular, etc. The particular mode of administration depends, at least in part, on the nature of the delivery vehicle employed for the vectors. In many embodiments, the vector or vectors are administered intravascularly, e.g. intraarterially or intravenously, employing an aqueous based delivery vehicle, e.g. a saline solution.

The amount of vector nucleic acid that is introduced into the animal is sufficient to provide for the desired excision and insertion of the exogenous nucleic acid into the genome. As such, the amount of vector nucleic acid introduced should provide for a sufficient amount of transposase activity and a sufficient copy number of the exogenous nucleic acid. The amount of vector nucleic acid that is introduced into the animal varies depending on the efficiency of the particular introduction or transfection protocol that is employed.

Following introduction of the vector DNA into the animal in combination with the requisite transposase, the nucleic acid region of the vector that is flanked by the transposase recognized insertion sequences of the vector, e.g., the vector nucleic acid positioned between the P element transposase recognized 31 base pair terminal repeats, is excised from the vector via the provided transposase and inserted into the genome of the animal. As such, introduction of the vector DNA into the animal is followed by subsequent transposase mediated excision and insertion of the exogenous nucleic acid carried by the vector into the genome of the animal.

Because of the particular properties of the subject transposon based vectors, the subject vectors may be used to integrate large pieces of DNA into the genome of the whole animal. Generally, the size of DNA that the vectors insert into the genome is at least about 50 bp, usually at least about 1000 bp and more usually at least about 2000 bp, where the size may be as large as 150,000 bp or larger. Where the vector inserts a large piece of DNA into the genome, the size of the inserted DNA ranges from about 30 to 150 kb.

The subject methods of stable integration of exogenous nucleic acid into the genome of a target animal find use in a variety of applications in which the stable integration of an exogenous nucleic acid into the genome of an animal is desired. Applications in which the subject vectors and methods find use include: research applications, polypeptide synthesis applications and therapeutic applications. Each of these representative categories of applications is described separately below in greater detail.

Research Applications

Examples of research applications in which the subject vectors find use include applications designed to characterize a particular gene. In such applications, the vector is employed to insert a gene of interest into an animal and the resultant effect of the inserted gene on the animal's phenotype is observed. In this manner, information about the gene's activity and the nature of the product encoded thereby can be deduced. The vectors can also be employed to identify and define DNA sequences that control gene expression, e.g. in a temporal (e.g. certain developmental stage) or spatial (e.g. particular cell or tissue type) manner. In such assays, the subject vectors are employed to stably integrate into the genome of a animal a selectable marker gene, e.g. antibiotic resistance, LacZ, etc., where the vector lacks a sufficient promoter for the marker the gene such that the marker is not significantly expressed, if at all, unless it is underneath an endogenous promoter element. If the marker gene is inserted into the animal's genome in sufficient relationship to an endogenous promoter sequence, it will be expressed. From the resultant expression profile of the marker gene, the endogenous promoter that is mediating its expression can then be characterized. Yet another research application in which the subject vectors find use is in the identification and characterization of the results of gene expression studies. For example, a plurality of animals with distinct integrated vectors are prepared in which the gene of interest is inserted into distinct locations in the genome, where expression of the gene of interest is dependent on endogenous promoter mediation, i.e. where the gene of interest lacks a promoter or is coupled to only a weak promoter. By plurality is meant at least two, where the number usually ranges from about 2 to 20,000, usually from about 2 to 200. This plurality of vector targeted animals may be produced by introducing the vector in a plurality of animals or taking a collection of pretargeted animals that are homogenous with respect to the insertion site of the gene, i.e. progeny of a single targeted animal, and then introducing transposase into one or more of the constituent members of the collection. The subject vectors can also be used to study integration mutants, where the vector is inserted randomly into the genome and disrupts a random gene whose identity and phenotype can be quickly characterized using standard techniques. One can also employ the subject vectors to produce models in which overexpression and/or misexpression of a gene of interest is produced in an animal and the effects of this mutant expression pattern are observed. One can also use the subject vectors to readily clone genes introduced into a host animal via insertional mutagenesis that yields phenotypes and/or expression patterns of interest. In such applications, the subject vectors are employed to generate insertional mutants through random integration of DNA. The phenotype and/or expression pattern of the resultant mutant is then assayed using any convenient protocol. In those mutants of interest, cloning of the DNA associated with the phenotype and/or expression pattern of interest is readily accomplished through use of the transposase recognized insertion sequences of the subject vector.

Polypeptide Synthesis Applications

In addition to the above research applications, the subject vectors also find use in the synthesis of polypeptides, e.g. proteins of interest. In such applications, a vector that includes a gene encoding the polypeptide of interest in combination with requisite and/or desired expression regulatory sequences, e.g. promoters, etc., (i.e. an expression module) is introduced into an appropriate animal to serve as an expression host for expression of the polypeptide (e.g., mouse, chicken, goat, and the like). Following introduction and subsequent stable integration into the animal's genome, the animal is then maintained under any necessary conditions (e.g., inducible system) sufficient for expression of the integrated gene. Once the transformed host expressing the protein is prepared, the protein is then purified to produce the desired protein comprising composition. Any convenient protein purification procedures may be employed, where suitable protein purification methodologies are described in Guide to Protein Purification, (Deuthser ed.) (Academic Press, 1990). For example, the protein could be purified from the milk of a goat with the appropriate attached signal sequences for expression and secretion into the milk. Then a lysate may be prepared from the milk and the protein further purified using HPLC, exclusion chromatography, gel electrophoresis, affinity chromatography, and the like.

Therapeutic Applications

The subject vectors also find use in therapeutic applications, in which the vectors are employed to stably integrate a therapeutic nucleic acid, e.g. gene, into the genome of a animal, i.e. gene therapy applications. The subject vectors may be used to deliver a wide variety of therapeutic nucleic acids. Therapeutic nucleic acids of interest include genes that replace defective genes in the target animal, such as those responsible for genetic defect based diseased conditions; genes which have therapeutic utility in the treatment of cancer; and the like. Specific therapeutic genes for use in the treatment of genetic defect based disease conditions include genes encoding the following products: factor VIII, factor IX, β-globin, low-density protein receptor, adenosine deaminase, purine nucleoside phosphorylase, sphingomyelinase, glucocerebrosidase, cystic fibrosis transmembrane regulator, α-antitrypsin, CD-18, omithine transcarbamylase, arginosuccinate synthetase, phenylalanine hydroxylase, branched-chain α-ketoacid dehydrogenase, fumarylacetoacetate hydrolase, glucose 6-phosphatase, α-L-fucosidase, β-glucuronidase, α-L-iduronidase, galactose 1-phosphate uridyltransferase, and the like. Cancer therapeutic genes that may be delivered via the subject vectors include: genes that enhance the antitumor activity of lymphocytes, genes whose expression product enhances the immunogenicity of tumor cells, tumor suppressor genes, toxin genes, suicide genes, multiple-drug resistance genes, antisense sequences, and the like. Because of the length of nucleic acid that can be carried by the subject vectors, the subject vectors can be used to not only introduce a therapeutic gene of interest, but also any expression regulatory elements, such as promoters, and the like, which may be desired so as to obtain the desired temporal and spatial expression of the therapeutic gene.

Kits

Also provided are kits for use in practicing the subject methods. The subject kits at least include a vector described above having an expression module sufficiently proximal to a transposase recognized insertion sequence, e.g., a P-foot, or a means for making the same, e.g., a provector having restriction endonuclease site, e.g., multiple cloning site, proximal to a transposase recognized insertion sequence and, optionally, a corresponding restriction endonuclease(s). The subject kits may further include other components that find use in the subject invention, e.g., buffers, delivery vehicles, etc.

The various components of the kit may be present in separate containers or certain compatible components may be precombined into a single container, as desired.

In addition to the above components, the subject kits will further include instructions for practicing the subject invention. These instructions may be present in the subject kits in a variety of forms, one or more of which may be present in the kit. One form in which these instructions may be present is as printed information on a suitable medium or substrate, e.g., a piece or pieces of paper on which the information is printed, in the packaging of the kit, in a package insert, etc. Yet another means would be a computer readable medium, e.g., diskette, CD, etc., on which the information has been recorded. Yet another means that may be present is a website address which may be used via the internet to access the information at a removed site. Any convenient means may be present in the kits.

The following examples are offered by way of illustration and not by way of limitation.

### Experimental

I. P Element Containing Vectors

Description of vectors used—The nucleic acid vectors used for this experimental section were based wholly or in part (restriction digested and subcloned DNAs) on the pCasper4, pBluescriptll, pcdna3.1, and delta2-3 (or related transposase producing vector). The constructs made were as follows: Restriction enzymes were used to replace the white gene in the pCasper4 vector with the CMV-bgal and SV40-neomycin resistance genes and this new construct will be referred to as C3.1. The next construct was created by removing the CMV-bgal gene from the C3.1 vector and this construct will be referred to as Cneo. Next the bgal gene and SV40 promoter were removed linking the CMV promoter to the neo resistance gene and this construct was called Ccmvneo. Next, the SV40-neo gene was removed from C3.1 and this construct was called Cbgal. Next, the CMV-bgal gene was replaced in the C3.1 vector with pBluescriptII and this construct was called Cbsneo. Finally, pBluescript was added into C3.1, between the CMV promoter and the 3′P foot and this construct was called Cbs3.1. Name txn units size genes promoters position of promoters

| | | Transcriptional | | | Promoter |
|---|---|---|---|---|---|
| Vector | Size | Units¹ | Promoters | Genes | Position |
| Casper4 | | 0 | N/A | N/A | N/A |
| C3.1 | | 2 | CMV, SV40 | Bgal, neo^{r} | CMV near |
| | | | | | P foot |
| Cneo | | 1 | SV40 | neo^{r} | SV40 near |
| | | | | | P foot |
| Ccmvneo | | 1 | CMV | neo^{r} | CMV near |
| | | | | | Pfoot |
| Cbgal | | 1 | CMV | Bgal | CMV near |
| | | | | | P foot |
| Cbsneo | | 1 | SV40 | neo^{r} | None near |
| | | | | | P foot |
| Cbs3.1 | | 2 | CMV, SV40 | Bgal, neo^{r} | None near |
| | | | | | P foot |

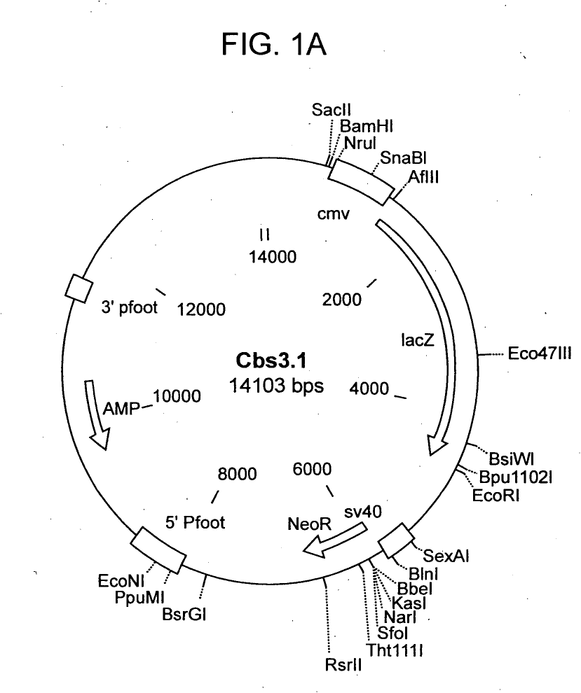

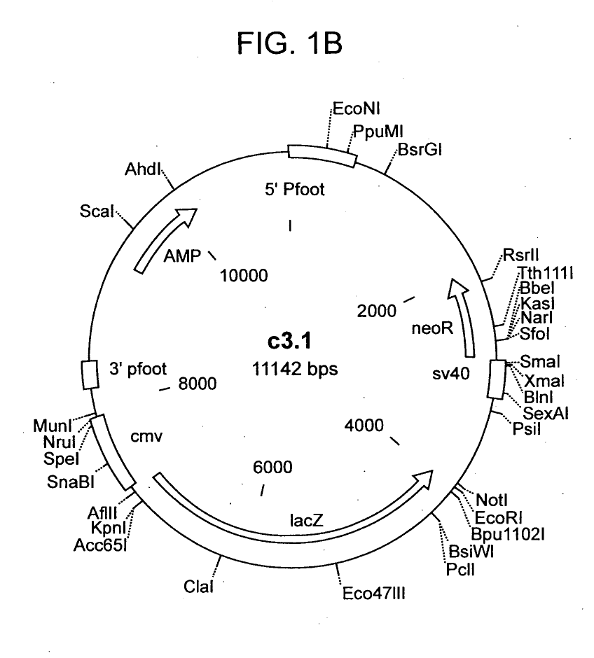

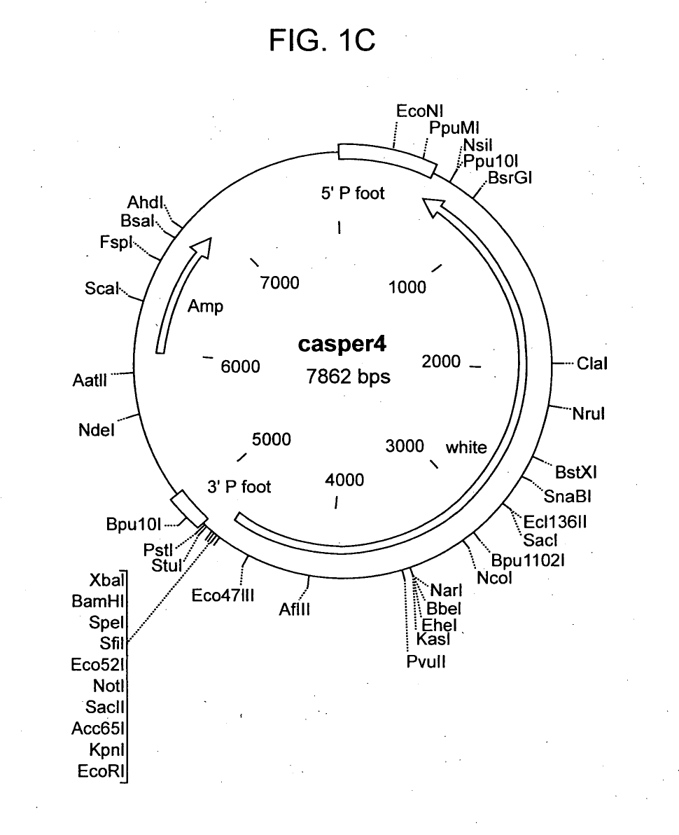

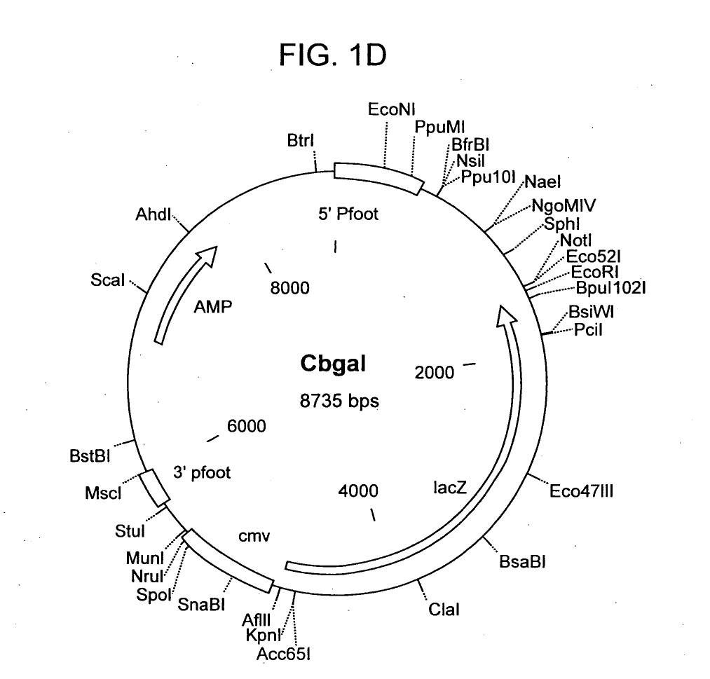

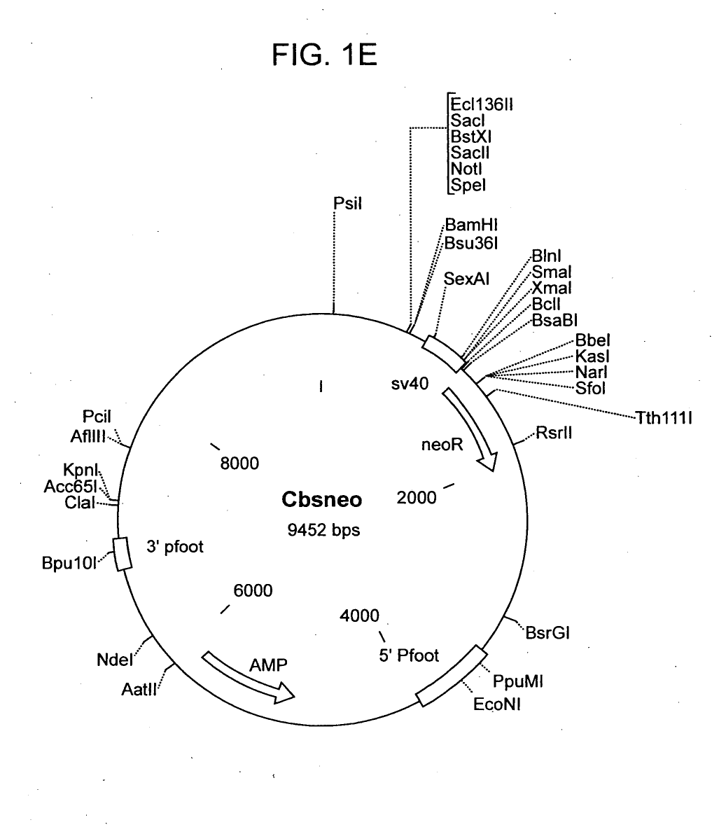

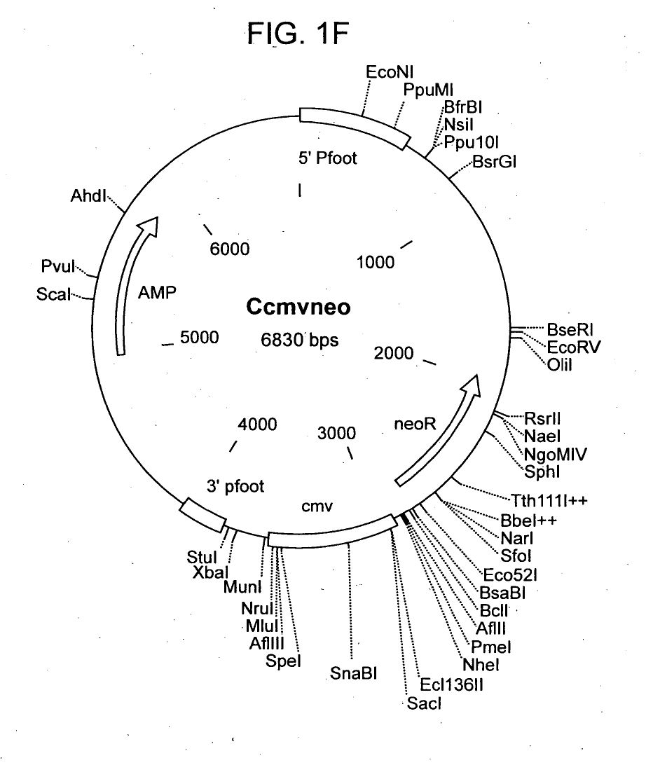

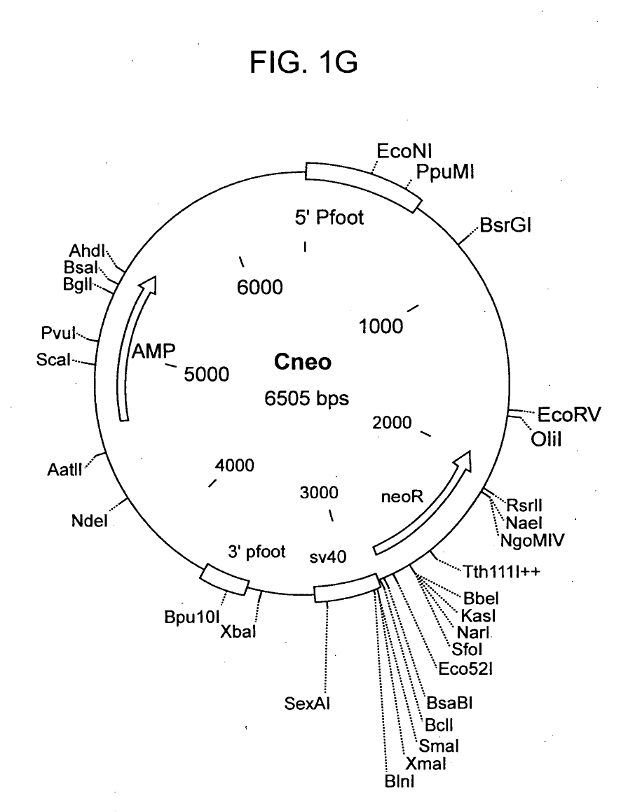

¹number of transcriptional units that express in vertebrates.
A diagram of each of the above vectors is provided in FIG. 1A and 1 G.

II. Integration in Animals is Dependent Upon Concentrations of Transposase and P Element Vector.
A. Strategy: Animals are co-injected with C3.1 and the transposase producing vectors. The injection empoyed is a systemic tail vein injection. Different mice receive different concentrations of each vector in order to determine a concentration that enables the C3.1 vector to integrate into the genome of the mouse. Concurrently, mice are also injected with just the transposase vector at the highest concentration (3 μg) to determine the time course for degradation. After degradation of the transposase producing vector any integrations are stable in the genome of the mouse. Analyses of the genomic DNA from these mice is then made.

B. Experiment: Male mice were injected twice, with a week in between each injection. Two months after the second injection, genomic DNA was harvested from a tail preparation for each mouse and analyzed. PCR assessment never detected the transposase vector. The PCR assessment of the C3.1 vector integrating into the genome occurred in a concentration dependent manner. That is, the more of the vector that integrates or the more of the vector that enables the integration in the injection mixture gave a stronger positive result (more integrations into the genome of the mouse). The results are provided in the following table.

| | Transposase Vector | C3.1 | PCR Results |
|---|---|---|---|
| Mouse Number | (μg) | (μg) | (Transposase/C3.1) |
| 1 | 0.5 | 1 | −/− |
| 2 | 1 | 1 | −/− |
| 3 | 3 | 1 | −/− |
| 4 | 0.5 | 3 | −/− |
| 5 | 1 | 3 | −/− |
| 6 | 3 | 3 | −/+ |
| 7 | 0.5 | 5 | −/+ |
| 8 | 1 | 5 | −/++ |
| 9 | 3 | 5 | −/++ |
| 10 | 0.5 | 10 | −/++ |
| 11 | 1 | 10 | −/++ |
| 12 | 3 | 10 | −/++ |

− no band
+ weak band
++ strong band

Rats and fish have also been examined for the integration of the C3.1 vector and the results are as seen above, modifying the amount of DNA based on the weight difference between animals.
III. Integration Occurs in Every Cell Type, Tissue Type and Organ Examined

Mouse #8 was used as the best positive with the least amount of DNA injected to determine the extent of gene delivery that occurred in the mouse. That is, we harvested a wide variety of tissues from the mouse and extracted the genomic DNA to ascertain the breadth of tissues that could have gene delivery via the C3.1 vector. The C3.1 vector was robustly detected in genomic DNA from every tissue examined, some of which included testis, liver, spleen, heart, lung, brain, intestine. This has been repeated with the same results on female mice.

IV. Integration can be Restricted to a Target Tissue

Male Mice and rats were injected into their testis. The volume of the injection was reduced to 30 microliters for mice and 100 microliters for rats. These animals gave transgenic offspring but did not show ubiquitous integration in the treated animal. Similar results were obtained localizing the distribution to either the female mammary gland or different sub-cutaneous points of injection. No transgenic offspring were observed from animals injected in a manner not exposing the animal's germ line to the integration vector.

V. Integrated Vectors are Heritable

Mice were injected as the #8 mouse in the table above and mated as described in the table below. All litters tested had at least one transgenic offspring. Southern blots and PCR analyses of the offspring indicated that typically 1 to 3 integrations occur per haploid genome for the injected concentration. The integrated DNA has been stable through four generations in mice. Analogous results were generated using rat matings. In addition, targeted injection into the specific organ and tissue type produces non-systemic integration. For example, injection into the testis directly showed heritablity, but no integrations were detected outside of the testis.

| | |
|---|---|
| Mating | % Transgenic |

| | | Time Post | Progeny (N, |
|---|---|---|---|
| Male | Female | Injection | #independent liters) |
| Injected ×2 | No Injection | 2 weeks | 25 (12, 2) |
| Injected ×1 | Injected ×1 | 1-2 days | 40 (20, 4) |
| Injected ×1 | Injected ×1 | 2 weeks | 38 (13, 2) |
| Injected ×1 | Injected ×1 | 5 weeks | 71 (14, 2) |
| Injected ×1 | Injected ×1 | 1 yr. | 44 (9, 2) |
| Control | Control | 1-2 days | 0 (19, 3) |
| Injected ×1 | Injected ×1 | | |

VI. One Actively Transcribed Gene in Proximity to at Least One of the P Feet is Essential for Animal Integration

The functional elements for integration into animals were examined. The two aspects that were important are that at least transcriptional active promoter is in the vector and that it is located near to a P foot. Though not wishing to be bound by any particular theory, this finding signifies that the DNA binding aspect of the promoter brings transcriptional factors into proximity of the P foot. As transcriptional factors relax the compaction of DNA in order to allow protein access, this may be the key step for transposase excision and integration. That is, any mechanism that relaxes the DNA in proximity to the P foot is likely to enable the transposase to integrate the P element into the genome of an animal.

| Vector | PCR results |
|---|---|
| Casper4 | − |
| C3.1 | + |
| Cneo | + |
| Ccmvneo | + |
| Cbgal | + |
| Cbsneo | − |
| Cbs3.1 | − |

VII. Genomes from Avian, Mice, Rats, Humans do not have Sequences that are Similar to the P Feet

PCR assessment for the prescence and absence of the 5′P foot and 3′P foot were in part used to detect integration of the vector into the genome of animals. During these experiments, it became clear that the hallmark sequences of the P element (the P feet) are not naturally present in non-fruitfly animals as no 5′P or 3′P feet sequences were detected in genomic DNA isolated from birds, mice, rats, and people.

VIII. Integration of a Large DNA Fragments into a Target Cell

P element vectors as described above have the capability to mobilize up to 150 kb of DNA. This presents the opportunity of the P element carrying a DNA sequence with therapeutic use of similar length into a target cell. The manipulation and handling of DNA fragments up to 4 megabases is difficult but has been reproducibly achieved. For example, this vector could allow the potential for analyzing genes on the size of the human huntington gene (˜150-200 kb).

IX. Other Transposons are Compatible with This Method

The beta-galactosidase (BGAL) gene was cloned into the sleeping beauty transposon. To directly compare efficiency of integration with the previous P element transposon results, the P element transposase gene was replaced by the Sleeping Beauty transposase gene (e.g., the same promoter was used to express Sleeping Beauty transposase as was used to express P element transposase). The Sleeping Beauty transposon containing the BGAL gene along with its transposase was injected systemically into the animal using the same criteria as for the P element above (e.g., IP or IV administration, 5-10 micrograms of transposon DNA, 1-2 micrograms of transposase DNA, etc.). The frequency of integration was less efficient than observed with the P element—one in twenty three progeny were transgenic (4%). However, it has been shown in cells and in vitro that this is also the case. In addition to detecting the germline transmission of the integrated Sleeping Beauty transposon, somatic integration was also detected (e.g., in genomic DNA isolated from liver, intestine and lung tissues of injected animals).

It is evident from the above results and discussion that the subject invention provides a novel nucleic acid vector having a broad range of applications. Advantages of the subject vector over other known nucleic acid vectors include the ability to integrate long stretches of nucleic acid into the genome of a population of target cells of a whole animal, i.e., to integrate the nucleic acid into the genome of a whole animal. This feature is advantageous for a number of reasons, including the ability to integrate an exogenous gene along with its native expression regulatory elements. Another feature of the subject invention is that the vectors provide for random insertion of a foreign nucleic acid, which is desirable in many applications. Yet another advantage of the subject invention is that the subject vectors do not elicit host immune reactions, in contrast with many viral based vectors. In addition, the subject vectors do not carry risk of viral infection or recombination hazards. As such, the subject vectors provide a number of advantages over other vectors currently known and employed in the art, and therefore represent a significant contribution to the art.

All publications and patent applications cited in this specification are herein incorporated by reference as if each individual publication or patent application were specifically and individually indicated to be incorporated by reference. The citation of any publication is for its disclosure prior to the filing date and should not be construed as an admission that the present invention is not entitled to antedate such publication by virtue of prior invention.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

1-10. (Canceled).

11. A method of inserting an exogenous nucleic acid into the genome of a non-Drosophilidae animal, said method comprising:
introducing into said animal a transposase recognized insertion sequence vector comprising said exogenous nucleic acid under conditions sufficient for transposition to occur so that said exogenous nucleic acid is inserted into said genome.

12. A method of inserting an exogenous nucleic acid into the genome of a non-Drosophilidae animal, said method comprising:
introducing into said animal a vector according to claim 1 under conditions sufficient for transposition to occur so that said exogenous nucleic acid is inserted into said genome.

13. The method according to claim 12, wherein said vector comprises a transposase domain.

14. The method according to claim 12, wherein said method further comprises introducing a second vector comprising a transposase domain into said animal.

15. The method according to claim 12, wherein said exogenous nucleic acid ranges in length from about 50 to 150,000 bp.

16. The method according to claim 12, wherein said target animal is a vertebrate.

17. The method according to claim 12, wherein said vertebrate animal is a mammalian animal.

18. The method according to claim 12, wherein said mammalian animal is a rodent.

19. A kit for use in inserting an exogenous nucleic acid into a target cell, said kit comprising:
a P-element vector comprising a pair of P-element transposase recognized insertion sequences flanking at least one transcriptionally active gene in proximity to at least one of the P-element transposase recognized isertion sequences.

20. The kit according to claim 19, wherein said transcriptionally active gene comprises a coding sequence that is expressed under intracellular conditions.

21. The kit according to claim 19, wherein said vector further comprises at least one endonuclease cleavage site positioned between said transposase recognized insertion sequences.

22. The kit according to claim 21, wherein said endonuclease cleavage site is present in a polylinker.

23-26. (Canceled)

27. A non-Drosophilidae animal or cells derived from said animal that has P-element transposase recognized insertion sequences integrated into the genome.

28. The animal or cells according to claim 27, wherein said animal is a vertebrate or said cells are vertebrate cells.

29. The animal or cells according to claim 28, wherein said animal is a mammal or said cells are mammalian cells.

30. The animal or cells according to claim 29, wherein said animal is a rodent or said cells are rodent cells.

31. A non-Drosophilidae animal or cells derived from said animal that have P element transposase recognized 31 bp insertion sequences integrated into the genome.

32. The animal or cells according to claim 31, wherein said animal is a vertebrate or said cells are vertebrate cells.

33. The animal or cells according to claim 32, wherein said animal is a mammal or said cells are mammalian cells.

34. The animal or cells according to claim 33, wherein said animal is a rodent or said cells are rodent cells.
